(19) United States
(12) Patent Application Publication
(10) Pub. No.: US 2010/0280147 A1
(43) Pub. Date: Nov. 4, 2010

(54) High refractive index interpenetrating networks for ophthalmic applications

(76) Inventors: Laura Hartmann, Berlin (DE); Curtis W. Frank, Cupertino, CA (US); Jaan Noolandi, Mountain View, CA (US); Donald T. H. Tan, Singapore (SG); Roger W. Beuerman, Singapore (SG)

Correspondence Address: LUMEN PATENT FIRM, 350 Cambridge Avenue, Suite 100, PALO ALTO, CA, 94306, US

(21) Appl. No.: 12/800,718

(22) Filed: May 19, 2010

Related U.S. Application Data
(63) Continuation-in-part of application No. 12/070,336, filed on Feb. 15, 2008.
(60) Provisional application No. 61/216,644, filed on May 19, 2009.

(51) Int. Cl. A61F 2/16

(52) U.S. Cl. 523/114

(57) ABSTRACT

Ophthalmic devices are provided based on an interpenetrating (IPN) double network hydrogel of a first network physically entangled with a second network. The first network is an entangled network of self-linked hydrophilic telechelic macromonomers and hydrophobic moieties. The second network is a hydrophilic network of crosslinked polyacrylic acid. The IPN double network hydrogels including the hydrophobic moieties are characterized by being optically clear and having refractive indices above 1.34.

### CROSS-REFERENCE TO RELATED APPLICATIONS

This application claims priority from U.S. Provisional Patent Application 61/216,644 filed May 19, 2009, which is incorporated herein by reference. This application is a Continuation-In-Part (CIP) of U.S. Non-Provisional patent application Ser. No. 12/070,336 filed on Feb. 15, 2008, which is incorporated herein by reference.

### FIELD OF THE INVENTION

The invention relates to hydrogels. In particular, the invention relates to interpenetrating double network hydrogels for ophthalmic applications.

### BACKGROUND OF THE INVENTION

Worldwide more than 10 million people suffer from blindness due to corneal diseases. However, only 100,000 corneal transplants are performed globally, due to lack of corneal donors and the potential risk of allograft rejection. The only alternative, keratoprothesis (KPro), has been available for many years, but their widespread application has been limited due to potential risk of serious complications.

To overcome these obstacles, a polymeric material, a so-called double network hydrogel or InterPenetrating Network (IPN) has been designed at Stanford University. This material conforms to the properties required in an artificial cornea such as high mechanical strength, physiological water content, enhanced resistance to protein adsorption and high oxygen permeability. The hydrogel is based on two interpenetrating polymer networks made from poly(ethylene glycol) (PEG) and poly(acrylic acid) (PAA), arguably the most biocompatible, protein-resistant and hydrophilic polymers available.

Though the major issue of corneal diseases is related to opacity, the cornea is also the first part of the eye that bends (or refracts) the light and provides most of the focusing power of the eye. To use the interpenetrating network hydrogel not only to replace the eye's natural cornea, but at the same time to correct the eye's existing refractive error, the material should have a high refractive index that is preferably tunable. Such a feature of the material would then also have a great potential to benefit 9% of the population suffering from hyperopia or presbyopia, the diminished ability of the eye to focus on objects.

Besides the use as an artificial cornea, the material could also be used as an intraocular lens or inlay, an implant in the eye changing the eye's optical power and making glasses or regular contact lenses redundant. Furthermore, a high refractive index IPN could be used as a new contact lens material.

However, present interpenetrating double network hydrogels have a refractive index that is (very) close to the refractive index of water (1.33), which is due to the high water content in the IPNs since the IPNs have been swollen with water. To make effective optical use of the IPNs a higher refractive index than water is needed. Accordingly, there is a need in the art to develop interpenetrating double network hydrogels with a high refractive index or a tunable refractive index. The present invention advances the art of IPNs by introducing an additional component to the first network of the IPNs, which result in a higher refractive index than IPNs without that component. The present invention further advances the art by introducing a tunable refractive index by adjusting the amount of the additional component.

### SUMMARY OF THE INVENTION

The present invention provides ophthalmic devices with a refractive index above 1.34 and optically clear which are suitable as an artificial lens, an inlay, an onlay, contact lens, or as an implant. The ophthalmic devices are based on an interpenetrating (IPN) double network hydrogel of a first network physically entangled with a second network. The principle teachings of the fundamental IPNs developed by Stanford University can be found e.g. in U.S. patent application Ser. Nos. 11/243,952 filed Oct. 4, 2005, 11/636,114 filed Dec. 7, 2006, 11/639,049 filed Dec. 13, 2006 and/or 12/070,336 filed Feb. 15, 2008, which are all incorporated herein by reference for all that they teach. In the IPN of this invention hydrophobic moieties are added to the first network creating an entangled first network of self-linked hydrophilic telechelic (i.e. self-linkable at both ends) macromonomers and hydrophobic moieties (self-linkable (macro)-monomers at one or both ends). The introduction of hydrophobic moieties to the hydrophilic telechelic macromonomers decreases the amount of water absorption capability by the IPN network. The IPNs including the hydrophobic moieties are characterized by having a refractive indice above 1.34. Tuning the refractive index of the IPN double network hydrogels, desirable for the particular application at hand, can be obtained by changing the water content capability of the first network and subsequently the IPN for example through changes in the amount (or type) of hydrophobic moieties or PEG telechelic macromonomers, and/or type or degree of self-linking in the first network (e.g. by changing the amount of photoinitiator in the polymerization of the first network).

The hydrophilic telechelic macromonomers in the first network (by virtue of being telechelic) can have covalent bonds to form individual macromonomer loops, to each other to form loops or networks of multiple macromonomers or to the hydrophobic moieties. It is noted that these bonds are self-linked without the need of additional or separate cross-linkers. In case there is a desire to increase the amount of linkage in the (first) network one could add a separate crosslinker to the polymerization process of the first network.

The second network is a hydrophilic network of crosslinked polyacrylic acid. It is preferred that the hydrophobic moieties are miscible with both the hydrophilic telechelic macromonomers and the polyacrylic acid. It is also preferred that the hydrophobic moieties are not UV-active in the IPN double network hydrogel after polymerization with the hydrophilic telechelic macromonomers.

In a preferred embodiment of the first network the hydrophilic telechelic self-linkable macromonomers are PEG-based macromonomers with a molecular weight of 3400 Da or higher. Examples of PEG-based macromonomers are poly(ethylene)glycol-diacrylate macromomoners or poly(ethylene)glycol dimethacrylate macromomoners. Examples of hydrophobic moieties are hexylacrylate (which has one-selflinkable end causing a reduction of the self-linking density in the first network) or PDMS-diacrylamide (two has two selflinkable ends).

### BRIEF DESCRIPTION OF THE DRAWINGS

FIGS. 1-2 show according to an exemplary embodiment of the present invention the formation of a first network PEG-Co-Hexylate-Hydrogel (FIG. 1) and the addition of a second network creating the formation of PEG-Co-Hexylate/Poly(Acrylic Acid) IPN (FIG. 2). The hydrophilic telechelic PEG macromonomers in the first network can have covalent bonds to form individual PEG macromonomer loops, to each other to form loops or networks of multiple PEG macromonomers or to hexylacrylate.

FIG. 3 shows according to an exemplary embodiment the present invention polymer content in dependence of the w % hexylacrylate added in the first network precursor solution.

FIG. 4 shows according to an exemplary embodiment of the present invention w % water content in dependence of the w % hexylacrylate added in the first network precursor solution.

FIG. 5 shows examples exemplary according to an embodiment of the present invention refractive index in dependence of the w % hexylacrylate added in the first network precursor solution. Examples of refractive index values (measured using a standard Abbe refractometer) are shown for a batch of PEG (4600Da)-co-Hexylate IPNs. In this example the refractive increases linearly with the addition of hexylacrylate to the first network. The invention is not limited to these exemplary results of refractive indices since different and/or higher values can be obtained or tuned by changing the water content, the amount (or type) of hydrophobic moieties, and/or type or degree of self-linking (e.g. by changing the amount of photoinitiator).

FIG. 6 shows according to an exemplary embodiment of the present invention the formation of polymerizable endgroups to PDMS.

FIGS. 7-8 show according to an exemplary embodiment of the present invention the formation of a first network PEG-Co-PDMS-Hydrogel (FIG. 7) and the addition of a second network creating the formation of PEG-Co-PDMS/Poly(Acrylic Acid) IPN (FIG. 8). The hydrophilic telechelic PEG macromonomers in the first network can have covalent bonds to form individual PEG macromonomer loops, to each other to form loops or networks of multiple PEG macromonomers or to PDMS-diMethacrylamide.

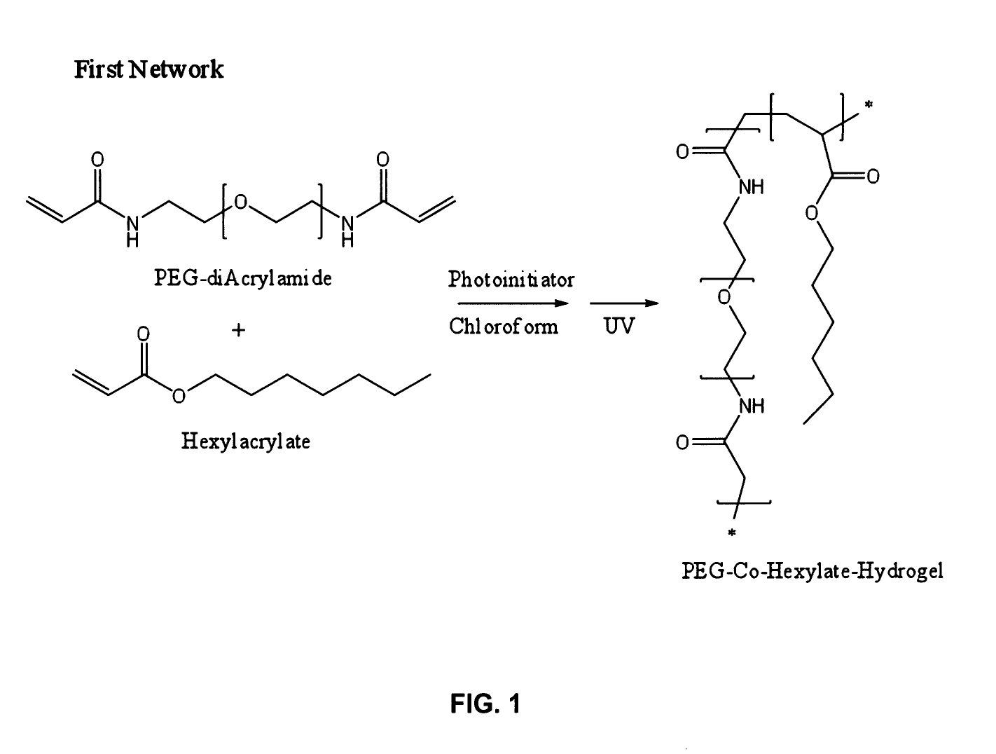

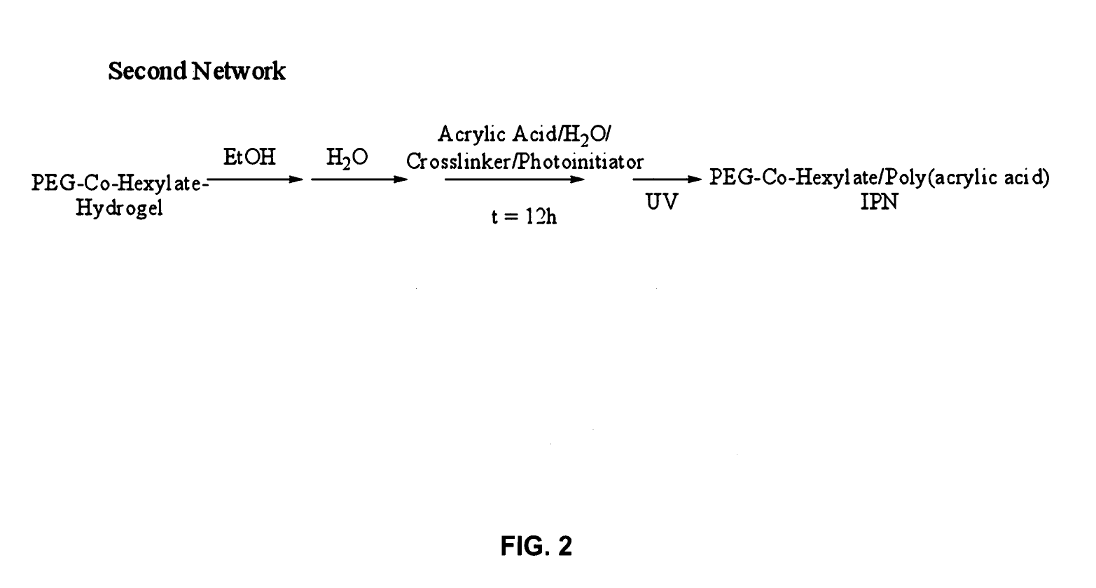

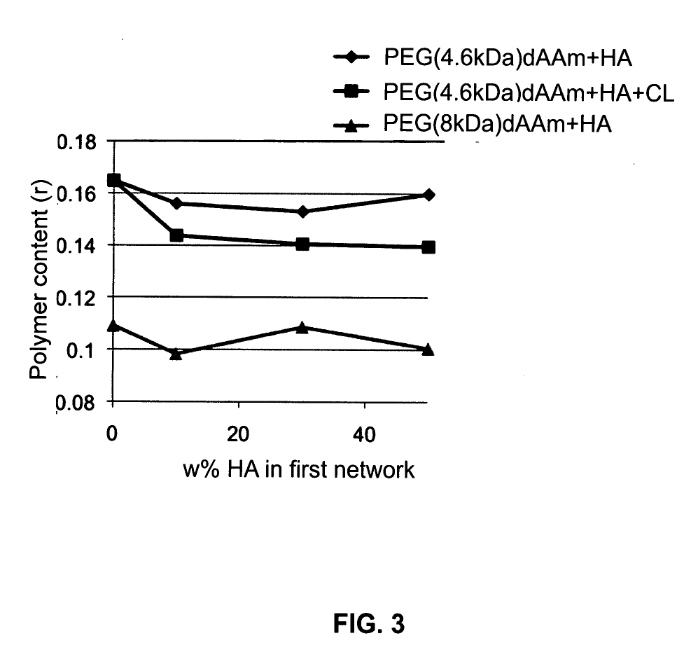

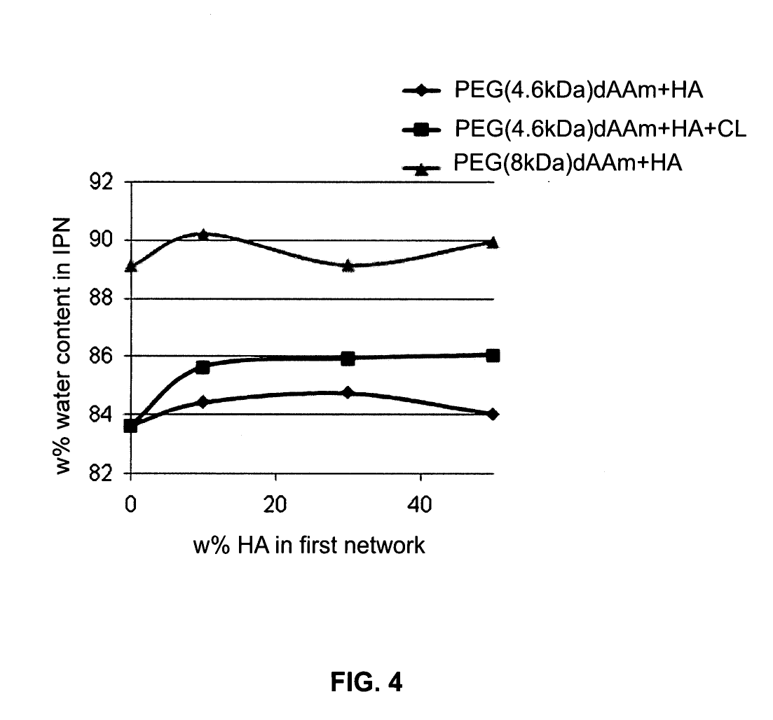

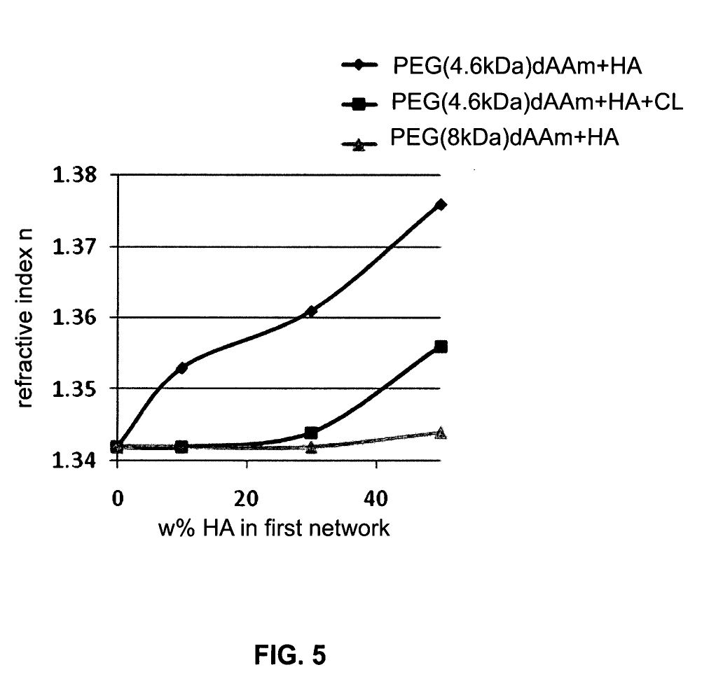

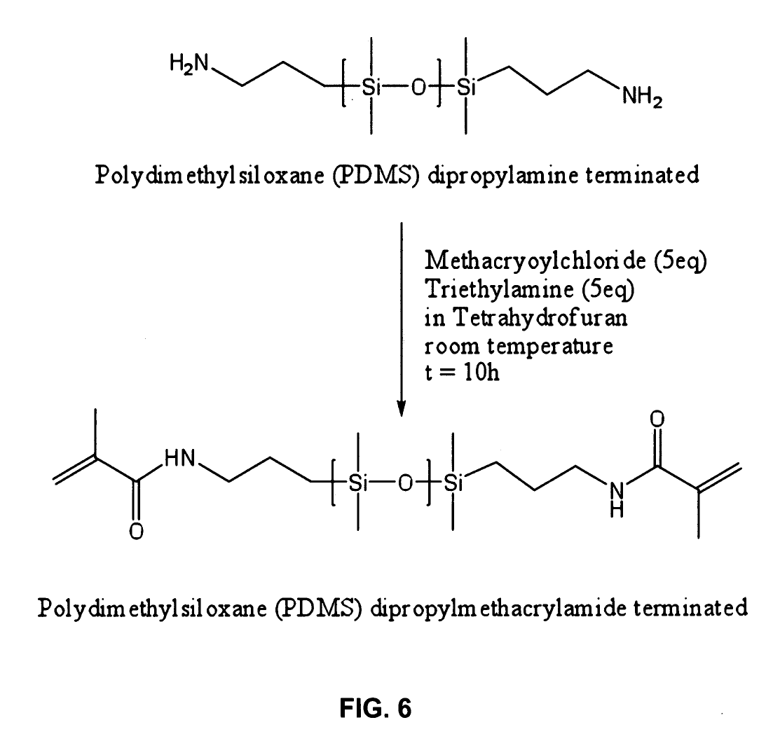

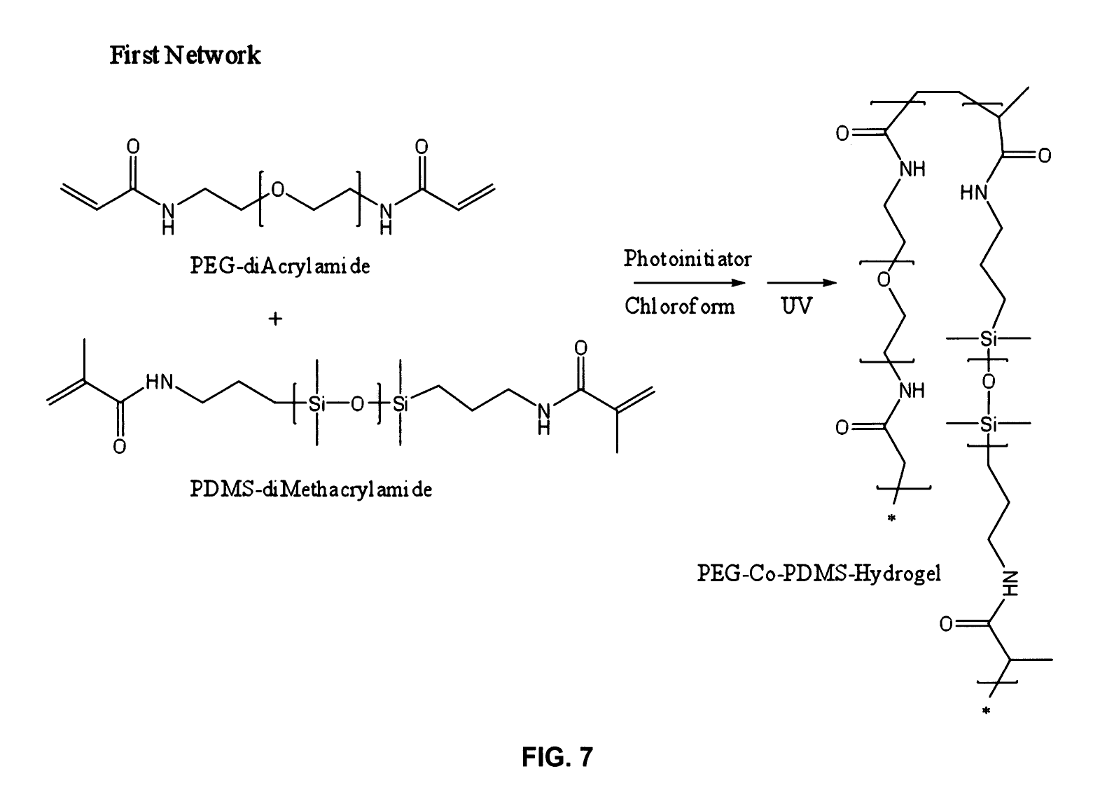

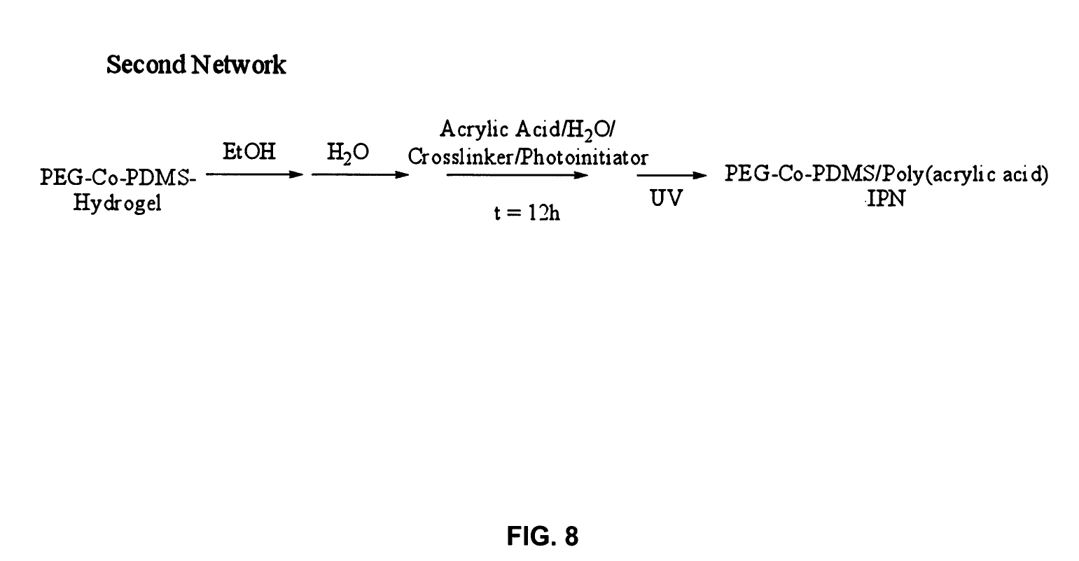

### DETAILED DESCRIPTION

To increase the refractive index of an interpenetrating double network hydrogel, water has to be excluded from the IPN double network hydrogel without limiting the optical transparency (clarity), the mechanical stability and biocompatibility of the material. The following description teaches examples of the invention where hydrophobic moieties ((macro)-monomers) that are miscible with the first (PEG)-telechelic network of macromonomers are introduced and are co-polymerized into the first (PEG)-network. Examples of these hydrophobic moieties are hexylacrylate and PDMS-dimethacrylamide which are polymerizable under the same conditions as the PEG-telechelic network (Photoinitiator, solvent, UV-reaction) and maintain optical transparency under in vivo conditions (buffered saline pH 7.4). In a second step, the second network of Poly(Acrylic Acid) is polymerized within the first network thus strongly enhancing mechanical properties of the material and increasing the water content to obtain sufficient nutrient and oxygen diffusion.

### Synthesis

### PEG-co-Hexylate-IPNs

PEG-diacrylamide or PEG-diacrylate macromonomers are synthesized as described in U.S. Ser. No. 12/070,336 filed on Feb. 15, 2008 and/or U.S. Ser. No. 12/148,534 filed on Apr. 17, 2008, which are both incorporated by reference for all that they teach. PEG-macromonomers are dissolved in Chloroform and Hexylacrylate is added to the solution. Examples of ratios are shown in Table 1, which are not limiting to the invention.

TABLE 1
IPNs with hydrophobic moieties

| | | | | | | | | | (CHCL₃) | PHOTOINITIATOR |
|---|---|---|---|---|---|---|---|---|---|---|
| MACROMONOMER | 0 W % | | 10 W % | | 30 W % | | 50 W % | | [ML] | [ML] |
| PEG(4.6 KDA)DAAM | 3 | G | 2.7 | G | 2.1 | G | 1.5 | G | 3 | 0.03 |
| HEXYLACRYLATE | 0 | ML | 0.34 | ML | 1.01 | ML | 1.7 | ML | | |
| PEG(8 KDA)DAAM | 2 | G | 1.8 | G | 1.4 | G | 1 | G | 2 | 0.02 |
| HEXYLACRYLATE | 0 | ML | 0.225 | ML | 0.675 | ML | 1.13 | ML | | |
| PEG(4.6 KDA)DAAM | 2 | G | 1.8 | G | 1.4 | G | 1 | G | 2 | 0.02 |
| PDMSDMAAM | 0 | G | 0.2 | G | 0.6 | G | 1 | G | | |
| PEG(4.6 KDA)DAAM | 3 | G | 2.7 | G | 2.1 | G | 1.5 | G | 3 | 0.03 |
| HEXYLACRYLATE | 0 | ML | 0.34 | ML | 1.01 | ML | 1.7 | ML | | |
| TRI-ETHYLENE- | 0 | ML | 0.016 | ML | 0.048 | ML | 0.080 | ML | | |
| GLYCODIMETHACRYLATE | | | | | | | | | | |

Then Photoinitiator Irgacure 2367 is added to the macromonomer solution. After casting the solution between two glass slides, the first network is polymerized by UV irradiation for 5 min. The first networks are then washed in Ethanol to wash out Chloroform, followed by washing in water to replace the Ethanol.

In a second step, the first network is soaked overnight in a solution of Acrylic Acid, water, Triethyleneglycoldimethacrylate as crosslinker and Photoinitiator (Irgacure 2367). UV-polymerization for 7 min leads to the formation of the double network or IPN. The IPNs are washed in PBS until a constant pH and salt concentration are reached.

PEG-co-Hexylate-IPNs with Constant Linking Density

The introduction of hexylacrylate decreases the overall (self)-linking density of the resulting networks as hexylacrylate is only mono-functionalized. To keep the linking density the same as for an IPN without hexylacrylate, a crosslinker, for example triethyleneglycol dimethacrylate, could be added to the mixture of the precursors forming the first network (exemplary ratios are shown in Table 1).

Since results have indicated that the choice of triethyleneglycol dimethacrylate crosslinker using an organic solvent for the precursor solution (here chloroform) leads to a higher linking of the hydrophobic moieties, but not the hydrophilic PEG-macromonomers, a different crosslinker with a higher overall hydrophilicity has to be used or the formation of the first network has to be performed in aqueous solution.

### PEG-co-PDMS-IPNs

To introduce PDMS moieties into the IPN double network hydrogel, PDMS-diaminopropyl terminated macromonomers have to be chemically modified by introducing polymerizable endgroups. Therefore the diamin-terminated PDMS can be reacted with Methacryloylchloride in THF overnight forming the PDMS-dimethacrylamide terminated macromonomer.

PEG-diacrylamide or PEG-diacrylate macromonomers are synthesized as described in U.S. Ser. No. 12/070,336 filed on Feb. 15, 2008 and/or U.S. Ser. No. 12/148,534 filed on Apr. 17, 2008, which are both incorporated by reference for all that they teach. PEG-macromonomer is dissolved in Chloroform and PDMS-dimethacrylamid is added to the solution. To mix both, the PEG-macromonomer and the PDMS-macromonomer, a surfactant has to be added. A different approach uses toluene instead of chloroform to obtain a clear precursor solution. Then Photoinitiator Irgacure 2367 is added to the macromonomer solution. After casting the solution between two glass slides, the first network is polymerized by UV irradiation for 5 min. The first networks are then washed in Ethanol to wash out Chloroform, followed by washing in water to replace the Ethanol.

In a second step, the first network is soaked overnight in a solution of Acrylic Acid, water, Triethyleneglycoldimethacrylate as crosslinker and Photoinitiator (Irgacure 2367). UV-polymerization for 7 min leads to the formation of the double network or IPN. The IPNs are washed in PBS until a constant pH and salt concentration are reached.

The invention has now been described with examples which are meant to be illustrative and should not be regarded as limiting to the scope of the invention as claimed.

What is claimed is:

1. An ophthalmic device, comprising: an interpenetrating double network hydrogel of a first network entangled with a second network, wherein said first network is an entangled network of self-linked hydrophilic telechelic macromonomers and hydrophobic moieties, wherein said hydrophilic telechelic macromonomers are covalently bonded to each other or to said hydrophobic moieties, and wherein said second network is a hydrophilic network of crosslinked polyacrylic acid.

2. The ophthalmic device as set forth in claim 1, wherein said hydrophilic telechelic macromonomers are PEG-based macromonomers.

3. The ophthalmic device as set forth in claim 2, wherein said PEG-based macromonomers are poly(ethylene)glycol-diacrylate macromomoners or poly(ethylene)glycol dimethacrylate macromomoners.

4. The ophthalmic device as set forth in claim 1, wherein said hydrophilic telechelic macromonomers have a molecular weight of 3400 Da or higher.

5. The ophthalmic device as set forth in claim 1, wherein said hydrophobic moieties are hexylacrylate or PDMS-diacrylamide.

6. The ophthalmic device as set forth in claim 1, wherein said interpenetrating double network hydrogel has a refractive index above 1.34.

7. The ophthalmic device as set forth in claim 1, wherein said interpenetrating double network hydrogel is optically clear.

8. The ophthalmic device as set forth in claim 1, wherein said hydrophobic moieties are miscible with both said hydrophilic telechelic macromonomers and said polyacrylic acid.

9. The ophthalmic device as set forth in claim 1, wherein said hydrophobic moieties are not UV-active in said interpenetrating double network hydrogel after said polymerization with said hydrophilic telechelic macromonomers.
